(19) United States
(12) Patent Application Publication
(10) Pub. No.: US 2009/0221688 A1
(43) Pub. Date: Sep. 3, 2009

(54) PHARMACEUTICAL COMPOSITIONS CONTAINING DOCETAXEL AND A DEGRADATION INHIBITOR AND A PROCESS FOR OBTAINING THE SAME

(75) Inventors: Antonio Machado, Juiz De Fora (BR); Aurelio Maranduba, Juiz De Fora (BR); Eneida Guimaraes, Juiz De Fora (BR); Livia Machado, Juiz De Fora (BR); Marcio Santiago, JR., Juiz De Fora (BR); Maria Silva, Juiz De Fora (BR)

Correspondence Address: BIRCH STEWART KOLASCH & BIRCH, PO BOX 747, FALLS CHURCH, VA, 22040-0747, US

(73) Assignees: Quiral Quimica DO Brasil S.A., Juiz de Fora (BR); Biorganica Ltda., Juiz de Fora (BR)

(21) Appl. No.: 12/162,772

(22) PCT Filed: Feb. 9, 2006
(86) PCT No.: PCT/BR2006/000016

§ 371 Date: Oct. 30, 2008

(30) Foreign Application Priority Data
Jan. 30, 2006 (BR) 20060012

(51) Int. Cl. A61K 31/337; A61P 35/00

(52) U.S. Cl. 514/449

(57) ABSTRACT

Pharmaceutical compositions containing polysorbate 80, anhydrous docetaxel (I) or its trihydrate and an organic acid with a pKa between 2.5-4.5 employed as a degradation inhibitor.

### SCOPE OF THE INVENTION

The present invention refers to pharmaceutical compositions, and means to obtain them, which are characterized by the use of a degradation inhibitor, in conjunction with an excipient, for the preparation of sterile and stable solutions containing anhydrous 4-acetoxy-2α-benzoyloxy-5β-20-epoxy-1,7-β-10-β-trihydroxy-9-oxo-tax-11-en-13α-il (2R,3S) 3-tert-butoxycarbonylamino-2-hydroxy-3-phenylpropionate ester, anhydrous docetaxel, (I) or its trihydrate. The solutions thus obtained exhibit improved stability and reduced levels of the principal degradation product, 4-acetoxy-2-α-benzoyloxy-5β-20-epoxy-1,7-α-10-β-trihydroxy-9-oxo-tax-11-en-13α-il (2R,3S) 3-tert-butoxycarbonylamino-2-hydroxy-3-phenylpropionate ester, 7-epi-docetaxel, (II).

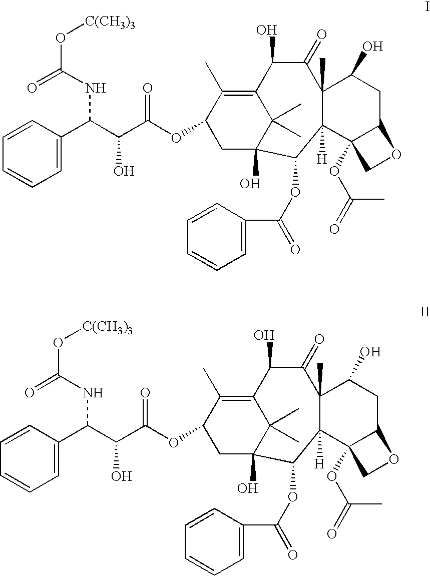

The first embodiment of the present invention relates to the obtention of highly stable pharmaceutical compositions, with a stability of at least 30 months when stored between 15-30° C., +19.

A second embodiment of the present invention relates to the fact that the principal degradation product, 7-epi-docetaxel (II), whose presence in the finished dosage forms containing docetaxel (T) or its trihydrate, is significantly reduced.

The formation of 7-epi-docetaxel (II) is sharply diminished. Its presence in the formulations obtained by way of the present invention being reduced to between 1 to 5% when compared to formulations described in the state of the art. This is realized by way of addition of a degradation inhibitor, ideally an organic acid with a pKa between 2.5 and 4.5 and/or an antioxidant.

The solutions described in the present invention are prepared by way of dissolution of the active ingredient (I) or its trihydrate, in a biocompatible vehicle, preferably polysorbate 80 treated with the degradation inhibitor, followed by filtration through a membrane with The porosity less than or equal to 0.22 μm followed by filling into adequate recipients.

In yet another embodiment of the present invention sterile solutions which are highly stable at room temperature, here defined as the range between 15-30° C.,±1°, as a function of the addition of at least one chemical agent which inhibits degradation of the active principle, and the formation of 7-epi-docetaxel (II).

### PRIOR ART

The active compounds 4-acetoxy-2-α-benzoyloxy-5β-20-epoxy-1,7-β-10-β-trihydroxy-9-oxo-tax-11-en-13α-il (2R,3S)
3-tert-butoxycarbonylamino-2-hydroxy-3-phenylpropionate ester, anhydrous docetaxel, (I) and its trihydrate are taxane derivatives obtained by chemical semi-synthesis and present anti-cancer and anti-leukemic proprieties. The above mentioned compounds have demonstrated pharmacological activity in various tumors and neoplasias.

U.S. Pat. No. 5,504,102 (Bristol-Myers Squibb Feb. 4, 1996), describes a process for the preparation of polyethoxylated castor oil (Cremophor EL® BASF) with low alkalinity by way of contacting the Cremophor EL with a bed of aluminum oxide . . . or by the addition of an acid, particularly, a mineral acid such as HCl or HNO₃, and the preparation of solutions of anti-neoplastic agents in this medium. In the context of the present invention, specifically when referring to docetaxel (I) or its trihydrate, the use of mineral acids is not efficient and even prejudicial, leading to the formation of other undesirable degradation products.

U.S. Pat. No. 5,698,582 (Rhone-Poulenc-Rorer of 16 Dec. 1997) describes a process for the preparation of compositions containing taxane derivatives in a surfactant, and the utility of the same to prepare perfusions. This patent does not contemplate the use of any acid. The solutions obtained before the preparation of the perfusion are not stable at room temperature for the shelf lives claimed within the scope of the present invention.

French patent FR 94 08479 (Rhone-Poulenc Rorer S.A.), describes a process for the preparation of the trihydrate of 4-acetoxy-2-α-benzoyloxy-5β-20-epoxy-1,7-β-10-β-trihydroxy-9-oxo-tax-11-en-13α-il (2R,3S) 3-tert-butoxycarbonylamino-2-hydroxy-3-phenylpropionate ester, utilizing recrystallization “in a mixture of water and an aliphatic alcohol containing 1 to 3 carbons, followed by drying the product obtained under determined conditions of temperature, pressure and humidity.” The patent claims the addition of ascorbic acid during the crystallization step of the docetaxel trihydrate. Nonetheless, this reference does not anticipate nor suggests an additional stability conferred to pharmaceutical formulations containing anhydrous docetaxel (I) or its trihydrate by way of the addition of an organic acid.

Patent pending PCT/BR/2004/000242 (Quiral Química do Brasil) claims processes, products and the use of the products in the treatment of infirmities utilizing the active principle (I) in acidified polysorbate 80.

Although the referred petition mentions obtaining compounds which form thermolabile hydrates which are only stable under refrigeration, in the prior document, no reference to improved stability with relation to time and temperature of storage, particularly that observed at room temperature, is foreseen. Additionally, no mention is made of the fact that the formation of the principal degradation product, 7-epi-docetaxel (IT), is drastically reduced in relation to the pharmaceutical formulations already described in the art.

### DETAILED DESCRIPTION OF THE INVENTION

The process and the products obtained by way of the present invention are advantageous with relation to those described in the state of the art, in that they demonstrate superior stability at room temperature, and the degradation of the active principle anhydrous 4-acetoxy-2-α-benzoyloxy-5β-20-epoxy-1,7-β-10-β-trihydroxy-9-oxo-tax-11-en-13α-il (2R,3S) 3-tert-butoxycarbonylamino-2-hydroxy-3-phenylpropionate ester, (I), or its trihydrate, to its epimer (II), is significantly reduced. The resulting pharmaceutical composition is of high purity, superior to that of similar preparations.

The chemical reactivity and the structure-activity relationships of the taxanes have been amply studied over the previous 25 years. For comprehensive reviews see Kingston, D. G. I. Trends Biotechnol. 1994, 12, 222.; Kingston, D. G. I. Recent Advances in the Chemistry and Structure-Activity Relationships of Paclitaxel. In Taxane Anticancer Agents Basic Science and Current Status, Goerg, G. I,; Chen, T. T.; Ojima, I.; Vyas, D. M., Eds.; ACS Symposium Series 583, American Chemical Society: Washington, D.C., 1995, pp. 203. and Gueritte-Vogelin, F.; Guenard, D; Dubois, J.; Marder, R.; Thoret, S.; and Potier, P. Chemistry and Biological Activity of Anti-tumor Taxoids, Advances in Natural Sciences, Vol. 2, No. 2, 2001, pp. 81-85. We hereby incorporate these publications by reference in that they contain pertinent information to the chemical transformations of the taxanes, as well as their structure activity relationships.

It is important to point out that docetaxel (I), and its trihydrate, as well as other taxanes, can suffer degradation under various conditions, with corresponding alterations, at times dramatic, in their activity and/or toxicity, for example, temperature, acidic and basic media, oxidizing and reducing agents, light as well as others. The principal known paths related in the state of the art are illustrated in FIG. I.

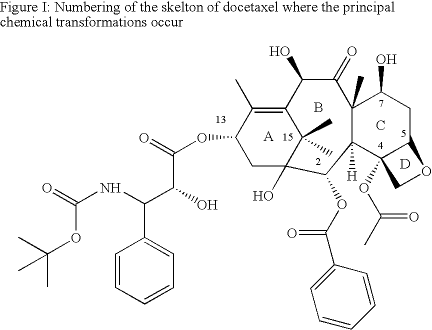

Degradation of Docetaxel:
in acidic media or in the presence of electrophilic agents, opening and/or rearrangement the D ring, as well as in the B ring is observed, depending on the conditions employed.
in basic media, cleavage of the ester groups at positions 2, 4 and/or 13 is observed.
one of the principal paths of degradation observed, be it in alkaline, neutral or strongly acidic media is the epimerization of the hydroxyl group at position 7 which results in the formation of 7-epi-docetaxel (II) by way of a retro aldol reaction.

The degradation of docetaxel can result in products which have reduced activity or are completely inactive. They also demonstrate pharmacological and toxicological profiles completely different from the active principle.

The importance of these complex transformations has grave consequences when considering the fact that the pharmaceutical formulations are destined for use in human subjects.

As an example, we cite the work of Bornique and Lemarié, Drug Metabolism and Disposition, Vol. 30, No. 11, pp. 1149-1152, 2002. In this study, the interactions of docetaxel (I) and its epimer 7-epi-docetaxel (II) with recombinant human cytochrome P450 1B1 (hCYP1B1) were investigated.

This cytochrome is present in various human tumors and is postulated to be responsible for the development of resistance of tumor cells toward chemotherapeutic agents, including docetaxel (I).

The authors observed that docetaxel was not metabolized by hCYP1B1 in vitro, employing the activity of 7-ethoxyresorufin O-desethylase (EROD activity) as a measure. However, at a concentration of 10 μM, the 7-epi-docetaxel (II) increased the activity of hCYP1B1 by more than 7 fold, confirming that (TI) is a potent inducer of this enzyme.

The consequence of this observation is that the authors of the present invention have identified that the presence of 7-epi-docetaxel (II) in pharmaceutical formulations or preparations made therefrom is a preponderant factor responsible for the development of resistance of tumor cells to the active principle, docetaxel (I) and/or its trihydrate, being therefore, desirable to minimize or eliminate the presence of 7-epi-docetaxel (II) in pharmaceutical preparations containing docetaxel (I) and/or its trihydrate. By means of the present invention, this objective has been achieved.

While the state of the art mentions the addition of ascorbic acid during the recrystallization of the active principle docetaxel trihydrate, a particularly innovative aspect of the present invention is the fact that it is advantageous to add at least one weak organic acid and/or antioxidant, in the preparation of pharmaceutical solutions of anhydrous docetaxel (I) or its trihydrate. This addition inhibits the epimerization to 7-epi-docetaxel (II) whose prejudicial effects have been previously exposed.

It was discovered, by way of real time stability testing, that the addition of a degradation inhibitor can increase the shelf-life of the finished dosage forms when stored at room temperature (15-30° C.) while at the same time inhibiting the epimerization of (I) and/or its trihydrate to 7-epi-docetaxel (II). The advantage of this result is evident, by increasing the shelf-life of the pharmaceutical preparations, and therefore the stability of the same, in addition to avoiding the formation of undesirable degradation products. Tables 1 and 2 demonstrate the results of these studies.

The degradation inhibitors that may be employed, include, but are not limited to, citric, tartaric, and ascorbic acids or other organic acids with a pKa between 2.5 and 4.5.

TABLE 1
Comparative study of the stability of solutions of anhydrous
docetaxel (I) in polysorbate 80 with the addition of various
organic acids and the respective concentrations of 7-epi-
docetaxel (II) formed as a function of time

| | Inhibitor employed | | | | | | | | | | | |
|---|---|---|---|---|---|---|---|---|---|---|---|---|
| | Inicial | | | | | | | | | | | |
| Concentration | concentration | | 3 months | | 6 months | | 12 months | | 24 months | | 30 months | |
| (mg/mL) | DCTX | 7-epi | DCTX | 7-epi | DCTX | 7-epi | DCTX | 7-epi | DCTX | 7-epi | DCTX | 7-epi |
| None | 40.01 | 0.10 | 39.67 | 0.12 | 38.12 | 0.53 | 37.48 | 1.36 | 36.25 | 2.31 | 35.87 | 3.01 |
| Acetic | 40.13 | 0.10 | 38.02 | 0.61 | 35.23 | 3.17 | — | — | — | — | — | — |
| Benzoic | 39.98 | 0.10 | 39.81 | 0.10 | 38.01 | 0.42 | 37.23 | 0.98 | 35.98 | 2.22 | — | — |
| Tartaric | 40.22 | 0.10 | 40.12 | 0.10 | 39.92 | 0.20 | 39.23 | 0.71 | 38.02 | 1.56 | 37.87 | 1.73 |
| Maleic | 39.76 | 0.10 | 39.54 | 0.14 | 38.57 | 0.26 | 37.92 | 1.21 | 36.21 | 2.01 | 35.98 | 2.67 |
| Citric | 40.54 | 0.10 | 40.34 | 0.11 | 39.99 | 0.23 | 39.12 | 0.87 | 38.01 | 1.81 | 37.68 | 1.92 |
| Ascorbic | 39.87 | 0.10 | 39.67 | 0.10 | 39.52 | 0.12 | 39.02 | 0.56 | 38.24 | 0.98 | 37.98 | 1.26 |
| Vitamin E | 39.76 | 0.10 | 37.12 | 1.42 | 34.78 | 2.67 | — | — | — | — | — | — |

TABLE 2
Comparative study of the stability of solutions of docetaxel
trihydrate in polysorbate 80 with the addition of various
organic acids and the respective concentrations of 7-epi-
docetaxel (II) formed as a function of time

| | Inhibitor employed | | | | | | | | | | | |
|---|---|---|---|---|---|---|---|---|---|---|---|---|
| | Inicial | | | | | | | | | | | |
| | concentration | | 3 months | | 6 months | | 12 months | | 24 months | | 30 months | |
| Concentration | DCTX | | DCTX | | DCTX | | DCTX | | DCTX | | DCTX | |
| mg/mL | 3H₂O | 7-epi | 3H₂O | 7-epi | 3H₂O | 7-epi | 3H₂O | 7-epi | 3H₂O | 7-epi | 3H₂O | 7-epi |
| None | 40.12 | 0.10 | 39.54 | 0.15 | 38.23 | 0.64 | 37.24 | 1.56 | 36.15 | 2.31 | 35.87 | 3.42 |
| Acetic | 40.14 | 0.10 | 38.12 | 0.61 | 35.03 | 3.31 | — | — | — | — | — | — |
| Benzoic | 39.87 | 0.10 | 39.75 | 0.12 | 37.74 | 0.32 | 37.11 | 1.08 | 35.25 | 2.43 | — | — |
| Tartaric | 40.04 | 0.10 | 40.01 | 0.11 | 39.89 | 0.19 | 39.01 | 0.82 | 37.99 | 1.36 | 37.87 | 1.78 |
| Maleic | 39.73 | 0.10 | 39.59 | 0.15 | 38.42 | 0.34 | 37.80 | 1.32 | 36.14 | 2.32 | 35.98 | 2.71 |
| Citric | 40.55 | 0.10 | 40.23 | 0.12 | 39.97 | 0.25 | 39.24 | 0.91 | 37.91 | 1.82 | 37.68 | 2.02 |
| Ascorbic | 39.97 | 0.10 | 39.42 | 0.10 | 39.32 | 0.13 | 39.01 | 0.71 | 38.02 | 1.02 | 37.98 | 1.32 |
| Vitamin E | 39.76 | 0.10 | 37.12 | 1.42 | 35.18 | 2.67 | — | — | — | — | — | — |

Observation 1: DCTX=anhydrous docetaxel; DCTX-3H₂O=docetaxel trihyrate; 7-epi=7-epi-docetaxel
Observation 2: All solutions were prepared by previously adjusting the pH of the polysorbate 80 with the respective acids to between 3.5 and 4.5. Anhydrous docetaxel (I) or its trihydrate were then solubleized to obtain a final concentration of 40 mg/mL, on an anhydrous base.
Observation 3: Samples were stored at 30±1° C. in clear vials of type II borosilicate glass.
Observation 4: Assay of (I) and (II) were determined by HPLC under the following analytical conditions: Column Spherisorb® RP 18 4.6×250 mm, particle size 5 μm; Mobile phase, gradient elution with Solution A Acetonitrile: H₂O (2:3 v/v), Solution B Acetonitrile, 100%. The gradient begins with 100% Solution A until 10% Solution A and 90% Solution B during 70 min. Flow 1.5 mL/min; Detection 227 nm; Loop 20 μL; Data are presented as area % without correction.
Observation 5: The acceptable limit adopted to define the stability of the pharmaceutical compositions was to “contain at least 90% of the amount declared on the label (40 mg/mL)”.

Upon examination of the data presented in Tables 1 and 2, it is evident that the addition of at least one degradation inhibitor, among those with characteristics proposed in the present invention, such as certain organic acids, exerts a profound effect on the stability of the composition as well as inhibits the formation of 7-epi-docetaxel (II). The best results were obtained with tartaric, citric and acorbic acids, which allowed storage for at least 30 months at a temperature of 30° C.±1° C. and with levels of 7-epi-docetaxel (II) significatively inferior to the composition without addition of any degradation inhibitor.

An experiment with Vitamin E demonstrated that the simple addition of an antioxidant as a degradation inhibitor is not sufficient to obtain the desired results. This fact, in conjunction with the observation that not all of the acids examined were adequate to obtain superior stability relative to that described in state of the art, demonstrates that, in order to obtain additional stability it is necessary to add one or more acid with unique characteristics. This is a result of the complex interaction between the components of the compositions, and involves factors such as pKa, redox potential, steric hindrance, nucleophilicity, solubility and reactivity.

### Example 1

### Preparation of a Pharmaceutical Composition Containing Anhydrous Docetaxel in Polysorbate 80 with the Addition of Tartaric Acid as a Degradation Inhibitor

In a beaker equipped with helical pneumatic agitation, under an atmosphere of nitrogen were added 100 mL of polysorbate 80 which was subsequently acidified with tartaric acid to obtain a pH of 3.9. This was followed by the slow addition of 4.00 g of anhydrous 4-acetoxy-2-α-benzoyloxy-5β-20-epoxy-1,7-β-10-β-trihydroxy-9-oxo-tax-11-en-13α-il (2R,3S) 3-tert-butoxycarbonylamino-2-hydroxy-3-phenylpropionate ester, and agitation was maintained until complete solubleization. The resulting solution was transferred to a pressurized vessel and filtered through a sterilizing membrane, 0.22 μm, in a sterile environment, under pressure, followed by filling in vials using habitual procedures. The solution thus prepared was stable as shown in Table 1 during 30 months when stored between 15-30±1° C.

### Example 2

### Preparation of a Pharmaceutical Composition Containing Anhydrous Docetaxel in Polysorbate 80 with the Addition of Citric Acid as a Degradation Inhibitor

In a manner similar to example 1, citric acid was employed with a resulting pH of 4.1. The solution thus prepared was stable as shown in Table 1 during 30 months when stored between 15-30±1° C.

### Example 3

### Preparation of a Pharmaceutical Composition Containing Anhydrous Docetaxel in Polysorbate 80 with the Addition of Ascorbic Acid as a Degradation Inhibitor

In a manner similar to example 1, ascorbic acid was employed with a resulting pH of 3.8. The solution thus prepared was stable as shown in Table 1 during 30 months when stored between 15-30°±1° C.

### Example 4

### Preparation of a Pharmaceutical Composition Containing Docetaxel Trihydrate in Polysorbate 80 with the Addition of Tartaric Acid as a Degradation Inhibitor

In a beaker equipped with helical pneumatic agitation, under an atmosphere of nitrogen were added 100 mL of polysorbate 80 which was subsequently acidified with tartaric acid to obtain a pH of 3.9. This was followed by the slow addition of 4.27 g of the trihydrate of 4-acetoxy-2-α-benzoyloxy-5β-20-epoxy-1,7-β-10-β-trihydroxy-9-oxo-tax-11-en-13α-il (2R,3S) 3-tert-butoxycarbonylamino-2-hydroxy-3-phenylpropionate ester, and agitation was maintained until complete solubleization. The resulting solution was transferred to a pressurized vessel and filtered through a sterilizing membrane, 0.22 μn, in a sterile environment, under pressure, followed by filling in vials using habitual procedures. The solution thus prepared was stable as shown in Table 2 during 30 months when stored between 15-30±1° C.

### Example 5

### Preparation of a Pharmaceutical Composition Containing Docetaxel Trihydrate in Polysorbate 80 with the Addition of Citric Acid as a Degradation Inhibitor

In a manner similar to example 4, citric acid was employed with a resulting pH of 4.1. The solution thus prepared was stable as shown in Table 2 during 30 months when stored between 15-30±1° C.

### Example 6

### Preparation of a Pharmaceutical Composition Containing Docetaxel Trihydrate in Polysorbate 80 with the Addition of Ascorbic Acid as a Degradation Inhibitor

In a manner similar to example 4, ascorbic acid was employed with a resulting pH of 3.8. The solution thus prepared was stable as shown in Table 2 during 30 months when stored between 15-30±1° C.

The examples cited are for illustrative purposes and should not be construed to limit the scope of the present invention.

1-15. (canceled)

16. A long life stable docetaxel pharmaceutical injectable concentrated composition comprising:
a quantity of docetaxel; a quantity of polysorbate 80 and a docetaxel anti-degradation agent or a mixture thereof in an amount effective to prevent the degradation by epimerization of said quantity of docetaxel and the formation of 7-epi-docetaxel, said anti-degradation agent selected from the group consisting of citric, tartaric, and ascorbic acids,
where this composition is sterile and stable at least 30 months when stored at a temperature between 15° and 30° C.,±1° C.

17. The composition of claim 16 wherein the concentration of docetaxel is about 36 to 44 mg/mL.

18. The composition of claim 16 containing at least about 968.4 mg to about 1345 mg of polysorbate 80.

19. The composition of claim 16 wherein the docetaxel anti-degradation agent is citric acid.

20. The composition of claim 19 containing at least about 1.93 mg to about 5.59 mg of citric acid.

21. The composition of claim 16 wherein the docetaxel anti-degradation agent is tartaric acid.

22. The composition of claim 21 containing at least about 1.93 mg to about 5.59 mg of tartaric acid.

23. The composition of claim 16 wherein the docetaxel anti-degradation agent is ascorbic acid.

24. The composition of claim 23 containing at least about 1.93 mg to about 5.59 mg of ascorbic acid.

25. A method of preparing the docetaxel pharmaceutical injectable concentrated composition of claim 16 comprising the step of adding the docetaxel anti-degradation agent to said quantity of docetaxel and polysorbate 80.

26. A method of preparing docetaxel pharmaceutical injectable concentrated composition of claim 16 comprising the step of adding said quantity of docetaxel and polysorbate 80, to the anti-degradation agent.

27. A method of preventing the formation of 7-epi-docetaxel in a quantity of docetaxel producing docetaxel pharmaceutical injectable concentrated long life stable compositions, the method comprising the steps of:
providing a quantity of docetaxel;
providing a quantity of polysorbate 80;
providing a docetaxel anti-degradation agent in an amount sufficient to prevent degradation by epimerization of said quantity of docetaxel and polysorbate 80, said anti-degradation selected from the group consisting of citric, tartaric, and ascorbic acids or a mixture thereof;
combining polysorbate 80 and docetaxel anti-degradation agent in an amount sufficient to acidifying the polysorbate 80 to prevent the degradation by epimerization of said quantity of docetaxel, and said quantity of docetaxel.

28. The method of claim 27 wherein the concentration of docetaxel is about 36 to 44 mg/mL.

29. The method of claim 27 wherein the amount of polysorbate 80 is from about 968.4 mg to 1345 mg.

30. The method of claim 27 wherein the amount of docetaxel anti-degradation agent is from about 1.93 mg to about 5.59 mg.
